SEQUENCE LISTING (4 sequences)

SEQ ID NO: 1 - protein, 243 aa, Homo sapiens
MGQTAGDLGWRLSLLLLPLLLVQAGVWGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGHSVWPLGFPTLSPQP

SEQ ID NO: 2 - protein, 229 aa, Homo sapiens
MTPQLLLALVLWASCPPCSGRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 3 - protein, 636 aa, Homo sapiens
MRGGRGAPFWLWPLPKLALLPLLWVLFQRTRPQGSAGPLQCYGVGPLGDLNCSWEPLGDLGAPSELHLQSQKYRSNKTQTVAVAAGRSWVAIPREQLTMSDKLLVWGTKAGQPLWPPVFVNLETQMKPNAPRLGPDVDFSEDDPLEATVHWAPPTWPSHKVLICQFHYRRCQEAAWTLLEPELKTIPLTPVEIQDLELATGYKVYGRCRMEKEEDLWGEWSPILSFQTPPSAPKDVWVSGNLCGTPGGEEPLLLWKAPGPCVQVSYKVWFWVGGRELSPEGITCCCSLIPSGAEWARVSAVNATSWEPLTNLSLVCLDSASAPRSVAVSSIAGSTELLVTWQPGPGEPLEHVVDWARDGDPLEKLNWVRLPPGNLSALLPGNFTVGVPYRITVTAVSASGLASASSVWGFREELAPLVGPTLWRLQDAPPGTPAIAWGEVPRHQLRGHLTHYTLCAQSGTSPSVCMNVSGNTQSVTLPDLPWGPCELWVTASTIAGQGPPGPILRLHLPDNTLRWKVLPGILFLWGLFLLGCGLSLATSGRCYHLRHKVLPRWVWEKVPDPANSSSGQPHMEQVPEAQPLGDLPILEVEEMEPPPVMESSQPAQATAPLDSGYEKHFLPTPEELGLLGPPRPQVLA

SEQ ID NO: 4 - protein, 918 aa, Homo sapiens
MLTLQTWLVQALFIFLTTESTGELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

(19) United States
(12) Patent Application Publication
(10) Pub. No.: US 2026/0193338 A1
(43) Pub. Date: Jul. 9, 2026

(54) USE OF IL-27 ANTAGONISTS FOR THE TREATMENT OF EBV-DRIVEN B LYMPHOPROLIFERATIVE DISEASES

(71) Applicants: INSTITUT NATIONAL DE LA SANTÉ ET DE LA RECHERCHE MÉDICALE, Paris (FR); UNIVERSITÉ PARIS CITÉ, Paris (FR); ASSISTANCE PUBLIQUE-HÔPITAUX DE PARIS (APHP), Paris (FR); FONDATION IMAGINE, Paris (FR); CENTRE NATIONAL DE LA RECHERCHE SCIENTIFIQUE, Paris (FR)

(72) Inventors: Sylvain LATOUR, Paris (FR); Emmanuel MARTIN, Paris (FR); Cécile GARCIN, Paris (FR)

(21) Appl. No.: 19/130,819

(22) PCT Filed: Nov. 20, 2023
(86) PCT No.: PCT/EP2023/082434

§ 371 Date: May 16, 2025

(30) Foreign Application Priority Data
Nov. 22, 2022 (EP) 22306715.8

(51) Int. Cl. C07K 16/24; A61P 35/00; C07K 16/28

(52) U.S. Cl. CPC C07K 16/244; A61P 35/00; C07K 16/2866

(57) ABSTRACT

Upon EBV infection, the inventors found that IL-27 is produced by infected B lymphocytes and IL27RAIL-27 interaction is required for in vitro maintenance and expansion of EBV-transformed B cells, potentially explaining the favorable outcome of the EBV viral disease in IL27RA-deficient patients. In addition, the inventors identified neutralizing anti-IL27 autoantibodies in individuals who developed sporadic infectious mononucleosis, thus possibly phenocopying the IL27RA deficiency. Collectively, these results demonstrate the critical role of IL27-IL27RA axis in immunity to EBV, but also the hijacking of this defense by EBV to promote expansion of infected cells. The IL27-IL27RA could therefore represent a novel therapeutic target to inhibit EBV-driven B lymphoproliferative diseases.

### FIELD OF THE INVENTION

The present invention is in the field of medicine, in particular oncology.

### BACKGROUND OF THE INVENTION

Epstein-Barr virus (EBV) is a gamma-herpes virus that infects most of humans and has a marked tropism for B lymphocytes. Importantly, EBV is known to be one of the strongest trigger of intrinsically uncontrolled B-cell proliferation and lymphomagenesis. Rare genetic diseases specifically predispose to defective control of EBV infection, leading to virus-associated hemophagocytic syndrome, lymphoproliferative disorders (LPD) such as Hodgkin and non-Hodgkin lymphomas. Selective vulnerability to EBV has been reported in association with inherited mutations impairing T cell immunity to EBV (Tangye, S. G. & Latour, S. Primary immunodeficiencies reveal the molecular requirements for effective host defense against EBV infection. Blood 135, 644-655, doi:10.1182 blood.2019000928 (2020).) Studies of these primary immunodeficiencies uncovered crucial pathways involved in T-cell response towards EBV-infected B lymphocytes and more generally in T-cell functions. In healthy individuals, efficiency of the immune response to EBV is indeed mainly dependent of the massive expansion of specific CD8⁺ cytotoxic T cells that eliminate EBV-infected B cells. For instance, in CTPS1, SH2DIA and MAGT1 deficiencies, CD8⁺ T-cell responses towards EBV-infected B lymphocytes are impaired as the result of defects in either cell-mediated cytotoxicity and/or expansion of specific cytotoxic CD8⁺ T cells. IL-27 is a two-chain cytokine, composed of EBI3 and IL-27p28 subunits belongs to the IL-12 family and signals through its heterodimeric receptor composed of gp130 and IL-27 receptor alpha (WSX-1) subunits. Several pieces of evidence indicated that IL-27 has a potent antitumor activity, related to the induction of tumor-specific Th1 and cytotoxic T lymphocyte (CTL) responses. However, the role of IL-27 in the susceptibility of EBV infection and the progression to lymphoma has never been investigated.

### SUMMARY OF THE INVENTION

The present invention is defined by the claims. In particular, the present invention relates to the use of IL-27 antagonists for the treatment of EBV-driven B lymphoproliferative diseases.

### DETAILED DESCRIPTION OF THE INVENTION

Epstein-Barr virus infection can engender severe B-cell lymphoproliferative disorders in immunocompromised individuals^{1,2}. In immunocompetent individuals, the primary infection is often asymptomatic or causing infectious mononucleosis, a self-limiting lymphoproliferative disorder. Selective vulnerability to EBV has been reported in association with inherited mutations impairing T cell immunity to EBV³. Herein, the inventors report bi-allelic loss-of-function mutations in IL-27RA in humans that underlie an acute and severe primary EBV infection but with a spontaneous favorable outcome. IL-27RA codes for the a subunit of the receptor of IL-27^{4,5}. In the absence of IL27RA, STAT1 and STAT3 phosphorylation in response to IL-27 is abolished in T cells of patients. IL-27 exerts a synergistic effect on TCR-dependent proliferation of T cells⁶ that is lost in patients cells, leading to impaired expansion of potent anti-EBV effector cytotoxic CD8+ cells. Upon EBV infection, the inventors found that IL-27 is produced by infected B lymphocytes and IL27RA-IL-27 interaction is required for in vitro maintenance and expansion of EBV-transformed B cells, potentially explaining the favorable outcome of the EBV viral disease in IL27RA-deficient patients. In addition, the inventors identified neutralizing anti-IL27 autoantibodies in individuals who developed sporadic infectious mononucleosis, thus possibly phenocopying the IL27RA deficiency. Collectively, these results demonstrate the critical role of IL27-IL27RA axis in immunity to EBV, but also the hijacking of this defense by EBV to promote expansion of infected cells. The IL27-IL27RA could therefore represent a novel therapeutic target to inhibit EBV-driven B lymphoproliferative diseases.

The first object of the present invention relates to a method of treating an EBV-driven B lymphoproliferative disease in patient in need thereof comprising administering to the patient a therapeutically effective amount of an IL-27 antagonist.

As used herein, the term “B lymphoproliferative disease” includes any type of leukemia or lymphoma of B cells. The term “B cell lymphoma” refers to a cancer that arises in cells of the lymphatic system from B cells. B cells are white blood cells that develop from bone marrow and produce antibodies. They are also known as B lymphocytes. B-cell malignancies include, but are not limited to, non-Hodgkin's lymphoma, Burkitt's lymphoma, small lymphocytic lymphoma, primary effusion lymphoma, diffuse large B-cell lymphoma, splenic marginal zone lymphoma, MALT (mucosa-associated lymphoid tissue) lymphoma, hairy cell leukemia, chronic lymphocytic leukemia, B-cell prolymphocytic leukemia, B cell lymphomas (e.g. various forms of Hodgkin's disease, B cell non-Hodgkin's lymphoma (NHL) and related lymphomas (e.g. Waldenstrom's macroglobulinaemia (also called lymphoplasmacytic lymphoma or immunocytoma) or central nervous system lymphomas), leukemias (e.g. acute lymphoblastic leukemia (ALL), chronic lymphocytic leukemia (CLL; also termed B cell chronic lymphocytic leukemia BCLL), hairy cell leukemia and chronic myoblastic leukemia) and myelomas (e.g. multiple myeloma). Additional B cell malignancies include small lymphocytic lymphoma, B cell prolymphocytic leukemia, lymphoplasmacytic lymphoma, splenic marginal zone lymphoma, plasma cell myeloma, solitary plasmacytoma of bone, extraosseous plasmacytoma, extra-nodal marginal zone B cell lymphoma of mucosa-associated (MALT) lymphoid tissue, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma/leukemia, grey zone lymphoma, B cell proliferations of uncertain malignant potential, lymphomatoid granulomatosis, and post-transplant lymphoproliferative disorder.

As used herein, the term “EBV-driven B lymphoproliferative disease” refers to a lymphoproliferative disease that is caused by Epstein-Barr virus (EBV).

As used herein, the term “treatment” or “treat” refers to both prophylactic or preventive treatment as well as curative or disease modifying treatment, including treatment of patients at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “IL-27” has its general meaning in the art and refers to a heterodimeric cytokine comprising the subunits p28 and EBI3.

As used herein, the term “p28” refers to the interleukin-27 subunit alpha. The term is also known as IL-27-A; IL27-A or interleukin-30. An exemplary amino acid sequence for p28 is represented by SEQ ID NO:1.

>sp|Q8NEV9|IL27A_HUMAN
Interleukin-27 subunit alpha
OS = Homo sapiens OX = 9606 GN = IL27
PE = 1 SV = 2
SEQ ID NO: 1
MGQTAGDLGWRLSLLLLPLLLVQAGVWGFPRPPGRPQLSLQELRR
EFTVSLHLARKLLSEVRGQAHRFAESHLPGVNLYLLPLGEQLPDV
SLTFQAWRRLSDPERLCFISTTLQPFHALLGGLGTQGRWTNMERM
QLWAMRLDLRDLQRHLRFQVLAAGFNLPEEEEEEEEEEEEERKGL
LPGALGSALQGPAQVSWPQLLSTYRLLHSLELVLSRAVRELLLLS
KAGHSVWPLGFPTLSPQP

As used herein the term “EBI3” refers to the Interleukin-27 subunit beta. The term is also known as IL-27 subunit beta; IL-27B, or Epstein-Barr virus-induced gene 3 protein (EBV-induced gene 3 protein). An exemplary amino acid sequence for EBI3 is represented by SEQ ID NO:2.

>sp|Q14213|IL27B_HUMAN
Interleukin-27 subunit beta
OS = Homo sapiens OX = 9606 GN = EBI3
PE = 1 SV = 2
SEQ ID NO: 2
MTPQLLLALVLWASCPPCSGRKGPPAALTLPRVQCRASRYPIAVD
CSWTLPPAPNSTSPVSFIATYRLGMAARGHSWPCLQQTPTSTSCT
ITDVQLFSMAPYVLNVTAVHPWGSSSSFVPFITEHIIKPDPPEGV
RLSPLAERQLQVQWEPPGSWPFPEIFSLKYWIRYKRQGAARFHRV
GPIEATSFILRAVRPRARYYVQVAAQDLTDYGELSDWSLPATATM
SLGK

As used herein, the terms “IL-27 receptor” or “IL-27R” refers to a heterodimeric receptor comprising IL-27 receptor, alpha subunit and gp130.

As used herein, the term “IL-27RA” has its general meaning in the art and refers to the interleukin-27 receptor subunit alpha. The term is also known as IL-27R subunit alpha; IL-27R-alpha; WSX-1, Cytokine receptor-like 1, Type I T-cell cytokine receptor (TCCR) or ZcytoR1. An exemplary amino acid sequence for IL-27RA is represented by SEQ ID NO:3. In particular, the extracellular domain of IL-27RA ranges from the amino acid residue at position 33 to the amino acid residue at position 516 in SEQ ID NO:3.

>sp|Q6UWB1|I27RA_HUMAN
Interleukin-27 receptor subunit
alpha OS = Homo sapiens OX = 9606
GN = IL27RA PE = 1 SV = 2
SEQ ID NO: 3
MRGGRGAPFWLWPLPKLALLPLLWVLFQRTRPQGSAGPLQCYGVG
PLGDLNCSWEPLGDLGAPSELHLQSQKYRSNKTQTVAVAAGRSWV
AIPREQLTMSDKLLVWGTKAGQPLWPPVFVNLETQMKPNAPRLGP
DVDFSEDDPLEATVHWAPPTWPSHKVLICQFHYRRCQEAAWTLLE
PELKTIPLTPVEIQDLELATGYKVYGRCRMEKEEDLWGEWSPILS
FQTPPSAPKDVWVSGNLCGTPGGEEPLLLWKAPGPCVQVSYKVWF
WVGGRELSPEGITCCCSLIPSGAEWARVSAVNATSWEPLTNLSLV
CLDSASAPRSVAVSSIAGSTELLVTWQPGPGEPLEHVVDWARDGD
PLEKLNWVRLPPGNLSALLPGNFTVGVPYRITVTAVSASGLASAS
SVWGFREELAPLVGPTLWRLQDAPPGTPAIAWGEVPRHQLRGHLT
HYTLCAQSGTSPSVCMNVSGNTQSVTLPDLPWGPCELWVTASTIA
GQGPPGPILRLHLPDNTLRWKVLPGILFLWGLFLLGCGLSLATSG
RCYHLRHKVLPRWVWEKVPDPANSSSGQPHMEQVPEAQPLGDLPI
LEVEEMEPPPVMESSQPAQATAPLDSGYEKHELPTPEELGLLGPP
RPQVLA

As used herein, the term “gp130” has its general meaning in the art and refers to the interleukin-6 receptor subunit beta. The term is also known as IL-6 receptor subunit beta; IL-6R subunit beta; IL-6R-beta; IL-6RB, CDw130, Interleukin-6 signal transducer, Membrane glycoprotein 130, Oncostatin-M receptor subunit alpha or CD130. An exemplary amino acid sequence for gp130 is represented by SEQ ID NO:4. The extracellular domain of gp130 ranges from the amino acid residue at position 23 to the amino acid residue at position 619 in SEQ ID NO:4.

>sp|P40189|IL6RB_HUMAN Interleukin-6
receptor subunit beta OS = Homo
sapiens OX = 9606 GNfIL6ST PE = 1 SV = 2
MLTLQTWLVQALFIFLTTESTGELLDPCGYISPESPVVQLHSNFT
AVCVLKEKCMDYFHVNANYIVWKTNHFTIPKEQYTIINRTASSVT
FTDIASLNIQLTCNILTFGQLEQNVYGITIISGLPPEKPKNLSCI
VNEGKKMRCEWDGGRETHLETNFTLKSEWATHKFADCKAKRDTPT
SCTVDYSTVYFVNIEVWVEAENALGKVTSDHINFDPVYKVKPNPP
HNLSVINSEELSSILKLTWTNPSIKSVIILKYNIQYRTKDASTWS
QIPPEDTASTRSSFTVQDLKPFTEYVERIRCMKEDGKGYWSDWSE
EASGITYEDRPSKAPSFWYKIDPSHTQGYRTVQLVWKTLPPFEAN
GKILDYEVTLTRWKSHLQNYTVNATKLTVNLTNDRYLATLTVRNL
VGKSDAAVLTIPACDFQATHPVMDLKAFPKDNMLWVEWTTPRESV
KKYILEWCVLSDKAPCITDWQQEDGTVHRTYLRGNLAESKCYLIT
VTPVYADGPGSPESIKAYLKQAPPSKGPTVRTKKVGKNEAVLEWD
QLPVDVQNGFIRNYTIFYRTIIGNETAVNVDSSHTEYTLSSLTSD
TLYMVRMAAYTDEGGKDGPEFTFTTPKFAQGEIEAIVVPVCLAFL
LTTLLGVLFCENKRDLIKKHIWPNVPDPSKSHIAQWSPHTPPRHN
ENSKDQMYSDGNFTDVSVVEIEANDKKPFPEDLKSLDLFKKEKIN
TEGHSSGIGGSSCMSSSRPSISSSDENESSQNTSSTVQYSTVVHS
GYRHQVPSVQVFSRSESTQPLLDSEERPEDLQLVDHVDGGDGILP
RQQYFKQNCSQHESSPDISHFERSKQVSSVNEEDFVRLKQQISDH
ISQSCGSGQMKMFQEVSAADAFGPGTEGQVERFETVGMEAATDEG
MPKSYLPQTVRQGGYMPQ

As used herein, the term “IL-27 activity” or “biological activity of IL-27” includes any biological effect of IL-27. In some embodiments, IL-27 activity includes the ability of IL-27 to interact or bind to a substrate or receptor. In some embodiments, the biological activity of IL-27 is the ability of IL-27 to stimulate the JAK/STAT pathway, predominantly STAT1 and STAT3 phosphorylation. In some embodiments, biological activity of IL-27 includes any biological activity resulting from IL-27 mediated signalling, in particular in EBV-infected cells such as B cells.

As used herein, the term “antagonist” is used in the broadest sense, and includes any molecule that partially or fully inhibits or neutralizes a biological activity of a polypeptide, such as IL-27, or that partially or fully inhibits the transcription or translation of a nucleic acid encoding the polypeptide. Exemplary antagonist molecules include, but are not limited to, antagonist antibodies, polypeptide fragments, oligopeptides, organic molecules (including small molecules), and anti-sense nucleic acids.

As used herein, the term “IL-27 antagonist” refers to a molecule that interacts with at least one factor selected from IL-27 heterodimer, p28, EBI3, IL-27 receptor (IL-27R) heterodimer, IL-27RA, and gp130, and inhibits IL-27-mediated signalling.

Exemplary IL-27 antagonists include antibodies that bind IL-27 heterodimer, antibodies that bind p28, antibodies that bind EBI3, antibodies that bind IL-27R heterodimer, antibodies that bind IL-27RA, IL-27RA extracellular domains (ECDs), and IL-27RA ECD fusion molecules. In some embodiments, an IL-27 antagonist is an antibody that binds to IL-27 heterodimer. In some embodiments, the IL-27 antibody that binds to the IL-27 heterodimer binds to p28 subunit of IL-27, but not to EBI3 subunit of IL-27. In some embodiments, the IL-27 antibody that binds to p28 but not EBI3 blocks binding of IL-27 heterodimer to IL-27R. In some embodiments, an IL-27 antagonist blocks binding of IL-27 to IL-27R.

As used herein, the term “antibody” has its general meaning in the art and refers to an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity. An antibody can be of any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The light chain includes two domains, a variable domain (VL) and a constant domain (CL). The heavy chain includes three (α, δ, γ) to five (μ, ε) domains, a variable domain (VH) and three to four constant domains (CH1, CH2, CH3 and CH4 collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site. CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDR set from each of a heavy and a light chain V region. Framework Regions (FRs) refer to amino acid sequences interposed between CDRs. The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35B (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system.

In some embodiments, the IL-27 antagonist is an IL-27R antibody.

As used herein, the term “IL-27 antibody” or “antibody that binds IL-27” refers to an antibody that binds to IL-27 heterodimer.

In some embodiments, an antibody that binds IL-27 inhibits IL-27-mediated signalling. IL-27 antibodies include antibodies that bind to the IL-27 heterodimer, but not to either p28 or EBI3 alone, antibodies that bind to p28 (alone and/or complexed with EBI3), and antibodies that bind to EBI3 (alone and/or complexed with p28). In some embodiments, an antibody binds to p28, but does not bind to EBI3. In some embodiments, an antibody binds to EBI3, but does not bind to p28. In some embodiments, an IL-27 antibody blocks binding of IL-27 to IL-27R. In one embodiment, the extent of binding of an anti-IL-27 antibody to an unrelated, non-IL-27 protein is less than about 10% of the binding of the antibody to IL-27 as measured, e.g., by a radioimmunoassay (RIA). In some embodiments, an antibody that binds to IL-27 has a dissociation constant (Kd) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸M to 10⁻¹³M, e.g., from 10⁻⁹M to 10⁻¹³ M).

In some embodiments, an anti-IL-27 antibody binds to an epitope of IL-27 that is conserved among IL-27 from different species.

In some embodiments, the IL-27 antagonist is a p28 antibody.

As used herein, the term “p28 antibody” or “antibody that binds p28” refers to an IL-27 antibody that binds to one epitope located in the extracellular domain of p28.

In some embodiments, an antibody that binds p28 inhibits IL-27-mediated signalling. A p28 antibody may bind to p28 alone, to p28 when it is complexed with EBI3, or both. In some embodiments, p28 antibody binds to p28 of IL-27 heterodimer, but does not bind to EBI3. In some embodiments, a p28 antibody prevents association of p28 with EBI3. In some embodiments, a p28 antibody blocks binding of IL-27 to IL-27R, as defined above.

In some embodiments, the p28 antibody is SRF388 that is a fully human IgG1 blocking antibody to IL-27. Upon administration, the p28 monoclonal antibody SRF388 targets and binds to p28, and inhibits the interaction of IL-27 with the IL-27 receptor subunit alpha (IL-27RA). This prevents the activation of IL-27RA and prevents IL-27-mediated signalling. This reduces signal transducer and activator of transcription 1 (STAT1) phosphorylation.

In some embodiments, the IL-27 antibody is a EBI3 antibody.

As used herein, the term “EBI3 antibody” or “antibody that binds EBI3” refers to an IL-27 antibody that binds to EBI3.

In some embodiments, an antibody that binds EBI3 inhibits IL-27-mediated signalling. An EBI3 antibody may bind to EBI3 alone, to EBI3 when it is complexed with p28, or both. In some embodiments, an EBI3 antibody prevents association of EBI3 with p28. In some embodiments, an EBI3 antibody blocks binding of IL-27 to IL-27R, as defined above.

In some embodiments, the IL-27 antagonist is an IL-27R antibody.

As used herein, the term “IL-27R antibody” or “antibody that binds IL-27R” refers to an antibody that binds to IL-27R heterodimer.

In some embodiments, an antibody that binds IL-27R inhibits IL-27-mediated signalling. IL-27R antibodies include antibodies that bind to IL-27R heterodimer, but not to either IL-27RA or gp130 alone, and antibodies that bind to IL-27RA (alone and/or complexed with gp130), and antibodies that bind to gp130 (alone and/or complexed with IL-27RA). In some embodiments, an IL-27R antibody blocks binding of IL-27 to IL-27R, as defined above.

In some embodiments, the IL-27 antagonist is an IL-27RA antibody.

As used herein, the term “IL-27RA antibody” or “antibody that binds IL-27RA” refers to an IL-27R antibody (as defined below) that binds to IL-27RA. In some embodiments, an antibody that binds IL-27RA inhibits IL-27 mediated signalling. A IL-27RA antibody may bind to IL-27RA alone, to IL-27RA when it is complexed with gp130, or both. In some embodiments, a IL-27RA antibody prevents association of IL-27RA and gp130. In some embodiments, a IL-27RA antibody blocks binding of IL-27 to IL-27RA, as defined above.

In some embodiments, the antibody of the present invention is a human, a humanized or a chimeric antibody.

In some embodiments, the IL-27 antagonist is an IL-27RA ECD fusion molecule

As used herein, the term “IL-27RA ECD fusion molecule” refers to a molecule comprising a IL-27RA extracellular domain (ECD), and one or more “fusion partners.” In some embodiment, the IL-27RA ECD and the fusion partner are covalently linked (“fused”). If the fusion partner is also a polypeptide (“the fusion partner polypeptide”), the IL-27RA ECD and the fusion partner polypeptide may be part of a continuous amino acid sequence, and the fusion partner polypeptide may be linked to either the N terminus or the C terminus of the IL-27RA ECD. In such cases, the IL-27RA ECD and the fusion partner polypeptide may be translated as a single polypeptide from a coding sequence that encodes both the IL-27RA ECD and the fusion partner polypeptide (the “IL-27RA ECD fusion protein”). In some embodiments, the IL-27RA ECD and the fusion partner are covalently linked through other means, such as, for example, a chemical linkage other than a peptide bond. Many known methods of covalently linking polypeptides to other molecules (for example, fusion partners) may be used. In other embodiments, the IL-27RA ECD and the fusion partner may be fused through a “linker,” which is comprised of at least one amino acid or chemical moiety. In some embodiments, the fusion partner polypeptide is an immunoglobulin constant domain (Fc region) to form an immunoadhesin. Immunoadhesins can possess many of the valuable chemical and biological properties of human antibodies. Since immunoadhesins can be constructed from a human protein sequence with a desired specificity linked to an appropriate human immunoglobulin hinge and constant domain (Fc) sequence, the binding specificity of interest can be achieved using entirely human components. The immunoglobulin sequence typically, but not necessarily, is an immunoglobulin constant domain. The immunoglobulin moiety in the chimeras of the present invention may be obtained from IgG1, IgG2, IgG3 or IgG4 subtypes, IgA, IgE, IgD or IgM, but typically IgG1 or IgG3. In some embodiments, the functional equivalent of the PD-1 or NRP-1 and the immunoglobulin sequence portion of the immunoadhesin are linked by a minimal linker.

As used herein, the term “therapeutically effective amount” refers to a sufficient amount of the IL-27 antagonist to treat the EB-driven lymphoproliferative disease. It will be understood, however, that the total daily usage of the agent is decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific agent; and like factors well known in the medical arts. For example, it is well within the skill of the art to start doses of the compound at levels lower than those required to achieve the desired therapeutic effect and to gradually increase the dosage until the desired effect is achieved. However, the daily dosage of the agent may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the agent for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Typically, the IL-27 antagonist of the present invention is combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. Typically, the pharmaceutical compositions contain vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Sterile injectable solutions are prepared by incorporating the active ingredient at the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The invention will be further illustrated by the following figures and examples. However, these examples and figures should not be interpreted in any way as limiting the scope of the present invention.

### FIGURES

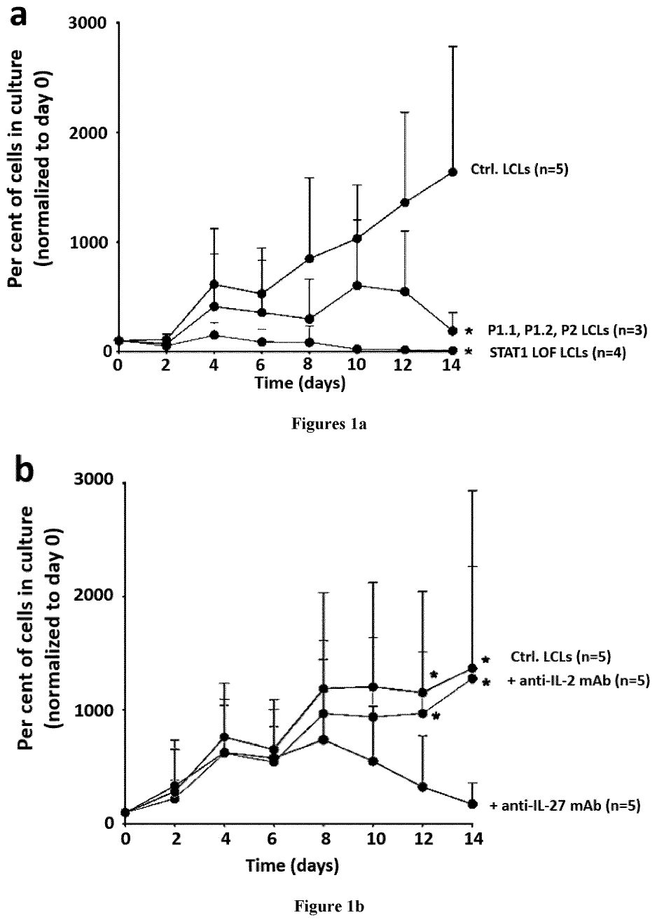

FIG. 1. EBV infection induces IL-27 production by B cells that is essential for their maintenance.
a, Curves of alive LCLs percentages cultured for 14 days. LCLs from three different controls, P1.1, P1.2 and P2 patients and four STAT1 LOF patients at different days of culture. Values are normalized to cell numbers at day 0. Data from FACS analysis of DAPI staining and cell counting of three independent experiments.
b, Curves of alive LCLs percentages as in (a) of three control LCLs cultured in presence or not of blocking anti-27 or anti-IL2 antibody. Values from three independent experiments.
(a) median±sd, two tailed Mann-Whiney tests (b) median±sd, paired t tests. *, P<0.05; **, P<0.01; ***, P<0.001; ****, P<0.0001.

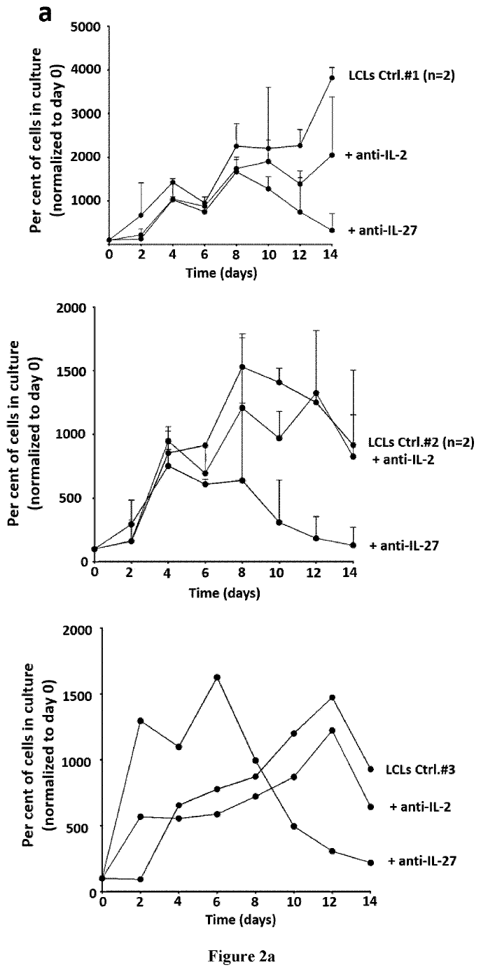

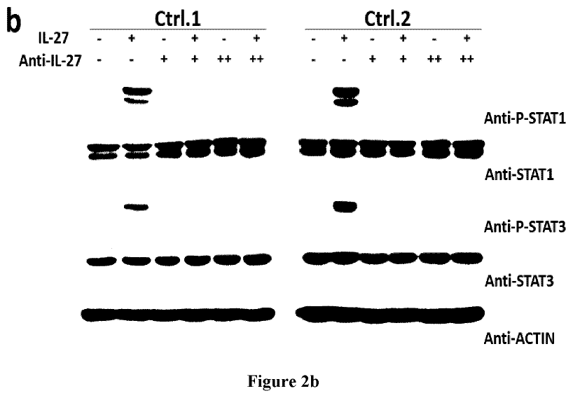

FIG. 2. Inhibition of proliferation and IL27RA signaling in LCLs by anti-IL-27 antibody.
a, Curves of percentages of alive LCLs cultured 14 days of three different controls (Ctrl. LCLs, in black) in presence of blocking anti-27 (in red) or anti-IL2 (in grey) antibody. For Ctrl. #1 and #2 curves of two independent experiments.
b, Immunoblots for phospho-STAT1, STAT1, phospho-STAT3, STAT3 and ACTIN expression in LCLs of two controls (Ctrl.1 and Ctrl. 2) stimulated with IL-27 (+) or not (−) in the presence or not of anti-IL27 blocking antibody at 2 μg·ml⁻¹ (+) or 20 μg·ml⁻¹ (++). Data from one representative experiment of two.

### EXAMPLE

### Methods

Human subjects and samples and study approval. The patient and the relatives studied here were living in and followed up in France. Informed and written consent was obtained from donors, patients and families of patients. The study and protocols conform to the 1975 declaration of Helsinki as well as to local legislation and ethical guidelines from the Comité de Protection des Personnes de l'Ile de France II and the French advisory committee on data processing in medical research. Experiments using samples from human subjects were conducted in France and Australia in accordance with local regulations and with the approval of the IRBs of corresponding institutions. The healthy controls were individuals who had not symptomatic EBV infection. Plasma and serum samples from the patients and controls were frozen at −80° C. immediately after collection.

### Patient Case Reports.

P1.1 was born from Caucasian consanguineous parents. Her past medical history was unremarkable unless 2 benign episodes of bronchiolitis and a short hospitalization for super-infection of a chickenpox skin lesion at 13 months old. At the age of 20 months, she was hospitalized for a 7-day persistent fever with sore throat. Physical examination showed hepatosplenomegaly (HSM) (respectively at 3 and 5 cm from the costal margin). Blood count showed remarkable hyper-leucocytosis (max 62G/L) with lymphocytosis (max 45G/L) and excess of basophil cells, mild anemia and normal platelets count. LDH was increased while bilirubin was normal. Triglycerides were increased but ferritin and fibrinogen were in the normal ranges. EBV serology was compatible with EBV primo-infection (anti VCA IgM and IgG positive, anti EBNA IgG negative), EBV PCR was positive between 3.56 and 4.33 log copies/mL. Peripheral immunophenotype showed sharp CD8+ lymphocytosis with activated profile. Blood smear displayed rare cytophagocytosis and excluded malignant disease. After 15 days, due to persistent symptoms (spiking fever, increased HSM) and significant biological abnormalities recapitulating incomplete hemophagocytosis (⅜ positive criteria), steroids (methylprednisolone 2 mg/kg). Partial response and development of hyper B-cell lymphocytosis prompted to start Rituximab, an anti-CD20 monoclonal antibodies (4 injections, 375 mg/m2 per week) allowing complete clinical and biologic normalization. Due to transient B-cell depletion, Immunoglobulin substitution was started for 12 months. Three years later, at the age of 5.5, partial B-cell deficiency was diagnosed because of absent anti-tetanus antibodies despite completed vaccination and persistent memory B cell lymphopenia. Due to recurrent upper respiratory tract infections, weekly sub cutaneous immunoglobulin substitution was restarted. At last follow up, at the age of 8 years, the patient is asymptomatic and is doing well. Her immunophenotype is in the normal range including memory B cells. Blood EBV viral load was low.

P1.2, the oldest brother was hospitalized at the aged of 8 months for severe mononucleosis compatible with primo-infection (positive anti-VCA IgM and IgG antibodies but negative anti-EBNA IgG) fever, HSM, lymphadenopathies, hyperleukocytosis (40G/L), mild increased of liver enzymes (AST 4N, ALT 7N and GGT 13N) and LDH (4N). He spontaneously improved within 2 weeks, without treatment. He presented benign bronchiolitis, one episode of pneumonitis in infancy and few episodes or upper respiratory infection during childhood. At last follow up, at the age of 18, he is healthy and well. His immunophenotype shows a mild CD4 and CD8 lymphopenia. Humoral immunity is normal (normal immunoglobulin dosage, positive post vaccinal response, normal memory B cells). Blood EBV viral load is negative. Two children of the family died in infancy (neonatal anoxia in a preterm baby and a 4 months boy due to dysmorphic syndrome and hypertrophic cardiomyopathy).

P2 was hospitalized at the aged of 17 years for severe mononucleosis with hepatitis and HLH compatible with a primary infection (blood EBV load at 5.56 log copies/mL, positive anti VCA IgM and IgG antibodies but negative anti EBNA IgG). She had no familial and medical previous histories. She presented with fever, sore throat, hepatic insufficiency, HSM, lymphadenopathies, bi-cytopenia with anemia (7.7 g/dL), hyperleukocytosis (40G/L), increased of liver enzymes (AST 3,5N, ALT 5,2N, GGT 22N and PAL 966UI/L) and LDH (4N). Triglycerides (7.25 g/L) and ferritin (1086 ng/mL) were increased and fibrinogen (1.32 g/L) decreased. The myelogram showed accumulation of macrophages with rare hemophagocytosis images. Lymphocyte immunophenotyping showed hyper-lymphocytosis (11808/mm³) with a high proportion of activated HLA-DR⁺ CD8⁺ T cells (70%). She was treated by corticosteroid therapy with methylprednisolone leading to a rapid improvement in few days. After corticosteroid therapy was stopped, she had relapse one month later characterized by hepatic cytolysis with a massive infiltration of T lymphocytes. One year and half later blood EBV load was strongly reduced to 2.95 log copies/ml. However, the serology remained abnormal with no detectable IgG anti-EBNA antibodies. Two and half years later, at the last follow up, she is well.

### Exome Sequencing and Analysis.

Exome capture and analysis were performed as previously described^{31,32}. The IL27RA variation identified in the patient (15:38803380 G/T), a homozygous substitution, c.286C>T (p.G96X) was reported in genome Aggregation database (gnomad.broadinstitute.org) at a 4·10⁻⁶ frequency, but never reported at a homozygous state.

### Sanger Sequencing.

Genomic DNA from peripheral blood cells of the patient, her parents, and brother was isolated according to standard methods. Oligonucleotide primers flanking the exon 3 of IL27RA gene were used to identify variants by genomic DNA amplication. PCR products were amplified using high fidelity Platinum TaqDNA Polymerase (Invitrogen) according to the manufacturer's recommendations, purified with the QIAquick gel extraction kit (Qiagen) and sequenced using the ABI PRISM BigDye Terminator Cycle Sequencing Ready Reaction Kit (PerkinElmer) according to the manufacturer's recommendations. All collected sequences were analyzed using 4peaks software (Version 1.8; A. Griekspoor and T. Groothuis, http://nucleobytes.com/index.php/4peaks).

### Cell Culture.

Peripheral blood mononuclear cells (PBMCs) collected from patients and healthy donors were isolated by Ficoll-Paque density gradient (Lymphoprep, Proteogenix) from blood samples using standard procedures. Expansion of T-cell blasts was obtained by incubating PBMCs for 72 h with phytohaemagglutinin (PHA) (2.5 μg/ml, Sigma-Aldrich) in Panserin 401 (Pan Biotech) supplemented with 5% human AB serum (Bio West), penicillin (100 U/ml) and streptomycin (100 μg/ml). After three days, dead cells were removed by Ficoll-Paque density gradient and blasts were maintained in culture with IL-2 (100UI/ml). Before to be tested in the various assays, phenotyping analyses of T-cell blasts were done for CD3, CD4, CD8, CD25, CD27, CD45RA and CD57 expression. Phenotypes of T-cell blasts from healthy donors and the patient were comparable for the expression of these different markers. EBV-transformed LCLs were obtained following procedures previously described (Izawa et al, 2017; Martin et al, 2014). LCLs of patients and controls were cultured in RPMI 1640 (Life Technologies) supplemented with 10% heat-inactivated fetal calf serum (Gibco), penicillin (100 U/ml) and streptomycin (100 μg/ml).

### EBV-Transformed LCLs and EBV-Specific T Cell Lines.

EBV-transformed LCLs were obtained following procedures previously described (Izawa et al, 2017; Martin et al, 2014). LCLs of patients and controls were cultured in RPMI 1640 (Life Technologies) supplemented with 10% heat-inactivated fetal calf serum (Gibco), penicillin (100 U/ml) and streptomycin (100 μg/ml). EBV-specific T cell lines were obtained from the patient and control healthy donors using PBMCs co-cultured with 45 Gy irradiated autologous LCLs at a PBMC/LCL ratio of 40:1. After 8-10 days, viable cells were stimulated with 45 Gy irradiated autologous LCLs at a PBMC/LCL ratio of 4:1. The cells were weekly re-stimulated with 45 Gy irradiated autologous LCLs in the presence of IL-2 (40 U/ml).

### EBV-Specific T Cell Detection.

HLA genotyping of the patient showed that he was a carrier of HLA-A*2, for which HLA-A*2 reagents were available to assess EBV-specific T cells. EBV-specific CD8⁺ T cells from PBMCs of the patient and a healthy control carrier of HLA-A*2 were detected using a mix of unlabeled EBV HLA-A2:01 Pro5 Pentamers (Proimmune) mixed with R-PE Pro5 Fluorotag in addition with BV785-anti-CD3, APC-anti-CD19, BV510-anti-CD4 and BV650-anti-CD8 antibodies according to the manufacturer's instructions. The EBV HLA-A2:01 Pro5 Pentamers mix contains 4 different pentamers presenting GLCTLVAML (residues 259-267 from BMLF-1), FLYALALLL (residues 356-364, from LMP-2), CLGGLLTMV (residues 426-434 from LMP-2) or YLLEMLWRL (residues 125-133 from LMP-1) peptides derived from BMLF-1, LMP-1 and LMP-2 proteins of EBV.

### Flow Cytometry.

Cell staining and the flow-cytometry-based phenotypic analyses of PBMCs and cells were performed according to standard flow cytometry methods. The following monoclonal antibodies conjugated to fluorescein isothiocyanate (FITC), R-phycoerythrin (PE), phycoerythrin-cyanin5 (PE-Cy5), phycoerythrin-cyanin5.5 (PE-Cy5.5), phycoerythrin-cyanin7 (PE-Cy7), Peridinin-chlorophyll (PerCP), Peridinin-chlorophyll-cyanin5.5 (PerCP-Cy5.5), allophycocyanin (APC), allophycocyanin-Cyanin7 (APC-Cy7), allophycocyanin-Vio7 (APC-Vio7), alexa-700, Brilliant Violet 421 (BV421), Brilliant Violet 510 (BV510), Brilliant Violet 605 (BV605), Brilliant Violet 711 (BV711), Brilliant Violet 650 (BV650) or Brilliant Violet 785 (BV785) were used: anti-CD3 (UCHT1), anti-CD4 (OKT4), anti-CD8 (RPA-T8), anti-CD11c (3.9), anti-CD14 (M5E2), anti CD16 (3G8), anti-CD19 (HIB19), anti-CD25 (BC96), anti-CD27 (LG.3A10), anti-CD28 (CD28.2), anti-CD31 (WM59), anti-CD45RA (H100), anti-CD45RO (UCHL1), anti-CD56 (HCD56), anti-CD57 (HNK-1), anti-CD70 (113-16), anti CD137 (4B4-1), anti-CD161 (HP-3G10), anti-CD183 (G025H7), anti-CD185 (J252D4), Anti-CD196 (G034E3), anti-CD197 (G043H7), and anti-CD279 (EH12.EH7), anti-CD303 (BDCA-2), anti-IgM (G20-127), anti-IgD (IA6-2), anti-CD355 (29A1.4), anti-TCRap (IP26), anti-TCRy6 (B1), anti-IgM (MHM-88), anti-IgD (IA6-2), anti-HLA-Dr (LN3), all purchased from BioLegend and anti-IL-27RA (FAB14791P) from R&D.

iNKT cells were detected by staining with anti-Vα24-Jα18 (6B11-BioLegend) and anti-Vβ11 (C21-Beckman Coulter). MAIT cells were detected by staining with anti-Vα7.2 (3C10-BioLegend) and anti-CD161 (HP-3G10-BioLegend) or using 5-OP-RU-loaded MR1 tetramer (NIH Tetramer Core Facility, Atlanta, GA).

For intracellular staining to phosphorylated-STAT1 and STAT3 proteins, cells were stimulated for 15 to 20 minutes with human recombinant IL-27 protein (Preprotech) (50 ng·mL⁻¹) and then fixed and permeabilized using Phosflow kit (BD biosciences) according to the manufacturer's instructions. Following antibodies were used: anti-phospho-ST AT 1 (pY701; clone 14/P-STAT-1) and anti-phospho-STAT3 (pY705; clone 4/P-STAT-3) both from BD biosciences.

All data were collected on LSR-Fortessa cytometer (from BD Biosciences) and analyzed using FlowJo Version 10.8.0 software (Tree Star).

### Cytokine Assays.

PBMCs were incubated with monoclonal antibodies against CD8, CD4, CD45RA, CCR7, CD127, and CD25. Naive and memory CD4⁺ and CD8⁺ T cells were isolated by first excluding Treg cells (CD4⁺CD45RA⁻CD25^{hi}CD127^{lo}) and then sorting CD4⁺ or CD8⁺ CD45RA⁺CCR7⁺ or CD45RA⁻CXCR5⁻CCR7^{+/-} cells, respectively. Isolated naive and memory CD4⁺ or CD8⁺ T cells were then cultured in 96-well round-bottomed (30-40×10³ cells/well) with T cell activation and expansion beads (coated with mAbs against CD2/CD3/CD28; Miltenyi Biotech) alone (Th0)/+IL-12 (Th1)/+IL-27 conditions for CD4 T cells or beads alone (Th0)/+IL-2/+IL-27 for CD8 T cells. After 5 d, supernatants were harvested and the production of IL-4, IL-5, IL-9, IL-10, IL-13, IL-17A, IL-17F, and IFN-γ was determined by cytometric bead arrays (Becton Dickinson); IL-22 secretion was measured by ELISA (eBioscience). For cytokine expression, activated CD4⁺ and CD8+ T cells were restimulated with PMA (100 ng/ml)/ionomycin (750 ng/ml) for 6 h, with brefeldin A (10 μg/ml) added after 2 h. Cells were then harvested, stained with extracellular markers and fixed and permeabilized using Cytofix/Cytoperm kit according to the manufacturer's instructions. Cells were then stained with specific antibodies to quantify intracellular levels of IL-4, IL-9, IL-13, IL-10, IL-17A, IL-17F, IL-22, IL-21, IFN-γ or IL-2, TNF-α, IFN-γ, Granzyme A, Granzyme B, Perforin in CD4 and CD8 T cells, respectively.

We assessed the secretion of IL-27 with ELISA kit (R&D Systems, #DY2526), according to the manufacturer's instructions. We also conducted intracellular cell staining to determine IL-27 expression in LCLs and different PBMCs subpopulations. The cells were incubated overnight with Monensin and Brefeldin A (Golgistop and Golgiplug, BD-Biosciences) after 2 hours of stimulation. Then, the cells were fixed/permeabilized using Cytofix/Cytoperm (BD-Biosciences) and labelled using anti IL-27 antibody and corresponding isotype antibody (R&D Systems, clone 307426) kit according to the manufacturer's instructions. All data were collected on LSR-Fortessa cytometer (from BD Biosciences) and analyzed using FlowJo Version 10.8.0 software (Tree Star).

### Proliferation Assay.

T-cell blasts or PBMCs were cultured respectively for 3 and 6 days in complete medium alone or with incremental doses of coated anti-CD3 antibody (clone OKT3 functional grade, eBiosciences) or anti-CD3+CD28 coated beads (Invitrogen) with or without adjunction of human recombinant IL-27 (PeproTech) at a concentration of 50 ng·ml⁻¹. Cell proliferation was monitored by labeling cells with the violet dye (Violet Proliferation Dye 450, BD Biosciences) prior to stimulation. EBV-transformed LCLs were washed 3 times in PBS and starved overnight in RPMI medium supplemented with 0.1% of foetal bovin serum. Then, LCLs were labeled with violet dye and cultured for 3 and 5 days in complete medium. After 3, 5 or 6 days of culture, cells were harvested and Violet dye dilution was assessed by flow cytometry. Proliferation and expansion indexes have been calculated using Flowjo software as: the total number of divisions/cells that went into division (for proliferation) and total number of cells/cells at start of culture (for expansion).

### Cytotoxicity Assay.

EBV-transformed LCLs of patient and healthy donor were infected with lentivirus containing pMSCV-CFP-DEVD-YFP construct (gift from P. Bousso, Pasteur Institut) which allows the expression of CFP and YFP protein linked by a caspase 3 cleavage sequence (DEVD)³³. 3 days after infection, infected cells were sorted using GFP reporter protein expression. Then, infected LCLs were coculture with autologous or HLA matched EBV specific CTL at different ratio of effector/target during 3 hours. Cell death of LCLs were quantify by FRET using flow cytometry analysis.

### Immunoblotting.

T cell blasts were stimulated for different times with human recombinant IL-27 protein (R&D) or anti-CD3/28 beads. The following antibodies were used for immunoblotting: anti-phosphorylated STAT1 (anti-phospho Y701, clone #D4A7), anti-phosphorylated STAT3 (anti-phospho Y705, clone #D3A7) and anti-ACTIN (clone #D3A7D18C11), anti-KU80 (clone #C48E7) purchased from Cell Signaling Technology, anti-IL-27RA (clone #191106) from R&D Systems. Membranes were then washed and incubated with anti-mouse or anti-rabbit HRP conjugated antibodies from Cell Signaling Technology. Pierce ECL western blotting substrate was used for detection.

### Plasmid Constructs, CRISPR-Cas9 Genome Editing and Infections.

A full length IL27RA cDNA was obtained by RT-PCR from blasts. The cDNAs were verified by sequencing and inserted into a bicistronic lentiviral expression vector encoding mCherry as a reporter (pLVX-EF1α-IRES-mCherry Vector, Clontech). Viral particles for infection were obtained by co-expression of the lentiviral vector containing IL-27RA with third-generation lentiviral plasmids containing Gag-Pol, Rev and the G protein of the vesicular stomatitis virus (VSVG) into BEK 293T using calcium phosphate. Viral supernatants were collected every 12 h on 2 consecutive days, starting 48 h after transfection, and viral particles were concentrated by ultracentrifugation at 49,000 g for 1.5 h at 12° C. The control and patient T-cell blasts were infected with viral particles at a minimal titer of 10⁸ TU ml⁻¹ (MOI 10) and 48 h after infection, cells were maintained in IL-2 in culture bis STAT phosphorylation or proliferation assays. For IL-27 gene knockdown by CRISP-cas9, the SpCas9(BB)-2A-eGFP (PX458) plasmid (plasmid no. 48138; Adgene) was used for genome editing. All single-guide RNAs (sgRNAs) were designed following a previously reported procedure³¹. Pairs of synthetized oligonucleotides were annealed, phosphorylated, ligated to linearized PX458 plasmid and transferred into Stabl3 bacteria (Thermo Fischer Scientific). Efficiency of sgRNAs was tested in HEK-293T cells. EBV-transformed LCLs of healthy donors transduced or not with lentiviral vector containing IL-27RA, were transfected 2 times by electroporation using Nepa21 electroporator (Nepagene, Japan) with PX458 plasmids, sorted on eGFP expression and maintained in culture with human recombinant IL-27 (100 ng·ml⁻¹). IL-27 expression and production was analyzed by intracellular flow cytometry and by ELISA in culture supernatants of LCLs respectively. Clones lacking IL-27 expression were sequenced for IL27 by Sanger sequencing using specific primers to determine the mutations that have been introduced by CRISPR Cas9 genome. To monitoring of survival of CRISPR IL27 or empty LCLs, IL-27 supplementation was stopped and cell death analysis was assessed by flow cytometry using Annexin V and DAPI labelling. All constructs were validated by Sanger sequencing using the BigDye™ Terminator v3.1 Cycle Sequencing Kit (Life Technologies) and a 3500xL Genetic Analyzer (Applied Biosystems) according to the manufacturer's instructions. Sequence analysis was performed using DNADynamo (BlueTractorSoftware).

### Anti-IL-27 Auto-Antibodies Detection.

ELISA was performed as previously described^{24,26}. In brief, 96-well ELISA plates (Maxisorp; Thermo Fischer Scientific) were coated by incubation overnight at 4° C. with 1 μg/ml rIL-27 (PeproTech). Plates were then washed (PBS-Tween 0.05%), blocked by incubation with the same buffer supplemented with 5% nonfat milk powder, washed, and incubated with serum or plasma samples (diluted 1/50) from patients and controls for 2 h at room temperature. Plates were thoroughly washed. Fc-specific HRP-conjugated IgG fractions of polyclonal goat antiserum against human IgG, IgA and IgM (Nordic Immunology Laboratories) were added to a final concentration of 2 μg/ml. Plates were incubated for 1 h at room temperature and washed.

Substrate was added, and optical density was measured (Victor X4™; Perkin Elmer). Then, the presence of anti-IL-27 auto-antibodies was confirmed by western blot according to the following method. 500 ng of human recombinant IL-27 (PeproTech) was separated by SDS-PAGE (10% acrylamide) under reducing conditions and transferred on PVDF membranes (Millipore). Membranes were blocked by incubation with PBS supplemented with 5% BSA and 0.05% Tween 20 and were washed, incubated overnight at 4° C. with plasma samples of patients or controls diluted 1/500 in PBS with 5% BSA and 0.01% Tween 20. Then, the membranes were washed 3 times and incubated for 1 h at room temperature with HRP-coupled anti human IgG, IgA and IgM secondary antibody (GAHu/Ig(Fc)/PO, Nordic MUbio), used at a final concentration. The membranes were washed 3 times and Pierce ECL western blotting substrate was used for detection.

### Neutralizing Assay.

The blocking activity of anti-IL-27 auto-Antibodies was determined by assessing STAT1 phosphorylation and T cell proliferation (see procedures below) in healthy control cells after stimulation with IL-27 (50 ng·mL⁻¹) or IL-27+anti-CD3 (OKT3; 0.1 μg/mL⁻¹) in the presence of 10% healthy control or patient plasma/serum. Anti-IL-27 commercial antibody (Ultra-Leaf™ purified anti-human IL27p28 Biolegend, clone MM27-7B) was used at 2 μg·ml⁻¹ concentration as positive neutralizing control.

LCLs growth monitoring assay. A method based on flow cytometry, which can allow cell numeration, has been developed for the evaluation of cellular proliferation. LCL cells were seeded at 125 000 cells/well in 96-well plates or at 1 million/mL in T25. Cells in the wells were counted every two days by flow cytometry using fluorescent beads (Precision Count Beads™ Biolegend cat:424902) following the provider's instructions. When specified, cells were cultured with antagonist blocking antibodies to IL-27 (Ultra-Leaf™ purified anti-human IL27p28 Biolegend, clone MM27-7B) or anti-IL-2 (Ultra-Leaf™ purified anti-human IL2 Biolegend, clone MQ1-17H12) at 2 and 3 μg·ml⁻¹ respectively every two days (three times a week).

IL27RA intracellular and surface staining. T-cell blasts were stimulated with anti-CD3/CD28 activating Dynabeads (LifeTechnologies) for 3 days. Surface IL-27RA were stained with purified anti-IL27RA antibody (R&D Systems, clone #191106) followed by Alexa Flour 488-conjugated secondary antibody (Invitrogen). Then, cells were fixed and permeabilized with a FOXP3 staining kit (eBioscience), and incubated with PE-conjugated anti-IL-27RA antibody (R&D Systems, clone #191106). Stained cells were analyzed with the BD LSRII-Fortessa (BD Biosciences), and data were processed with FlowJo software (Tree Star).

### Immunofluorescence.

T-cell blasts were stimulated with anti-CD3/CD28 activating Dynabeads (LifeTechnologies) for 3 days. Surface IL-27RA were stained with anti-IL27RA antibody (R&D Systems, clone #191106) followed by Alexa Flour 488-conjugated secondary antibody (Invitrogen). Then, cells were fixed with 4% paraformaldehyde, permeabilized with 0.1% Triton X-100, and incubated with anti-IL-27RA antibody (R&D Systems, clone #191106) followed by Alexa Flour 546-conjugated secondary antibody (Invitrogen). Cell nuclei were stained with 49-6-Diamidino-2-phenylindole. Images were acquired with a Leica SP8 STED confocal microscope (Leica Microsystems).

### Statistical Analysis.

P values were calculated with Student unpaired or paired t-test or Mann-Whitney test with a two-tailed distribution. A P value less than 0.05 is considered significant.

### Results

### Identification of Bi-Allelic Mutations in IL27RA Causing Loss-of-Function in Three Patients With Severe Primary EBV Infection

Infectious mononucleosis is a benign condition triggered by primary EBV infection that mainly occurs in adolescents and adults. We investigated three children (P1.1, P1.2 and P2) from two families who had severe primary Epstein-Barr virus infection. In family 1, P1.1 and P1.2 developed unusual early onset infectious mononucleosis (IM) symptoms at the age of 20 and 8 months in respectively, while in P2 from family 2, the primary infection occurred at the age 17 years (data not shown). Severe EBV infection in the patients was characterized by persistent fever, hepatitis, splenomegaly and manifestations of HLH requiring hospitalization and treatment with anti-CD20 (P1.1) or corticosteroids (P2) with relapse. In addition, patients had a previous history during their first years of life of ear, nose and throat infections (P2), bronchiolitis (P.1.2) and severe VZV infection (P1.1). In P1.1, high blood loads of EBV (up to 1×10⁵ EBV copies/mL) persisted without any clinical symptoms, for more than 12 months. No clinical manifestations were noted thereafter in P1.1, P1.2 and P2 (6, 17 and 3 years post IM, respectively). Immunological phenotyping of P1.1, P1.2 and P2 PBMCs showed counts and proportions of the different leukocyte subpopulations within normal ranges with the exception of decreased fractions of memory effector CD8⁺ and CD4+T cells (data not shown). In P1.1 and P2, memory CD27+ B cell counts were also decreased. Ig levels were normal in patients, excepted for IgA that was low in P1.1. T-cell proliferation to PHA and anti-CD3 antibody was normal for P1.1, P1.2 and P2. Based on these features, a genetic defect leading to an immunodeficiency associated with vulnerability to EBV infection was suspected in these patients.

To identify a potential gene defect responsible for the disease in the three patients, we performed whole exome sequencing (WES) in P1.2 and P2. Genetic variants identified by WES were filtered based on their combined annotation dependent depletion/mutation significance cutoff (CADD/MSC) scores and their allele frequencies in Gnomad and in the exome data bases of our institute. Significant genetic variants in the IL27RA gene were retrieved in both P1.2 and P2, consisting of a homozygous premature stop codon (g.19:14150387C>T (rs375317876), c.286C>T; p.Gln96X) in P1.2 and two heterozygous compound variants in P2, including a missense mutation (g.19:14160060C>G (rs201107107), c.1336C>G, p.Arg446Gly) and an essential acceptor splice mutation (g.19:14159791A>C (rs778365769), c.1142-2A>C). All three variations are located in exons coding for the extracellular domain of the protein encoded by IL27RA and are predicted to have a high impact using CADD/MSC scoring (MSC of 3.3 for IL27RA) and have a low allele frequency (AF) in gnomAD and in our own data base (p.Gln96X, CADD: 33, gnomAD AF: 7.96·10⁻⁶; p.Arg446Gly, CADD: 8.7, gnomAD AF: 5.84·10⁻⁴; c.1142-2A>C, CADD: 19.1, gnomAD AF: 7.96·10⁻⁶). The segregation of the IL27RA variants was examined in members of both families by Sanger sequencing (data not shown). Both parents in family 1 were heterozygous for the g.19:14150387C>T and the affected brother (P1.2) was as expected homozygous. In family 2, mother and father were heterozygous carriers of the c.1336C>G variant and the c.1142-2A>C, respectively. These data were consistent with an autosomal recessive inheritance of the disease. The p.Gln96X premature stop codon is predicted to remove most of the protein that contains extracellular, transmembrane and intracellular domains (data not shown). The splice mutation was found to cause abnormal splicing characterized by the usage of an alternative acceptor site within exon 9 leading to an in frame deletion of 45 nucleotides resulting in a shorter protein lacking 15 amino-acids (p.Gln381_Ala395del) (data not shown).

IL-27RA codes for the α subunit of IL-27 receptor that, with gp130, forms an heterodimer receptor belonging to the type 1 group 2 of cytokine receptor family^{5,7}. The only known ligand for this receptor is the IL-27 cytokine. IL-27RA is mostly expressed in lymphoid tissues, specifically thymus, spleen, lymph node and peripheral blood leukocytes^{7,8}. IL27RA protein expression was assessed in T cells of patients (data not shown). To determine this impact of the identified genetic variations in patients, IL27RA expression was examined in T cells. IL27RA was found to be upregulated in activated control T cells in response to CD3/CD28 stimulation, whereas no or very weak IL27RA expression was detected on the surface of activated T cells from P1.1, P1.2 and P2 (data not shown). IL27RA expression was also absent in T-cell extracts from P1.1 and P1.2 as shown by western blot contrasting with the detectable amounts of IL27RA in control cell extracts (data not shown), while in P2, IL27RA protein was apparent but most of the detected protein had a significant lower molecular weight (data not shown). This lower molecular weight is likely explained by abnormal post-translational modifications such as cleavage and/or abnormal glycosylation resulting of the two mutations p.Arg446Gly and p.Gln381_Ala395del (data not shown). Of note, although the IL27RA mutant protein was not expressed at the surface of P2 cells, it was detectable intracellularly by flow cytometry and immunohistochemistry showing IL27RA accumulation in the cytoplasm. T cells from both parents of P2 showed intracellular IL27RA staining and decreased IL27RA surface staining, consistent with their heterozygous status and indicating that the two mutations have deleterious consequences (data not shown).

IL-27 when bound to the gp130-IL27RA heterodimer is known to activate the JAK/STAT pathway, predominantly STAT1 and STAT3. This activation is mediated by the gp130 intracytoplasmic domain that contains binding sites for JAK1/2^{4,5,8}. The ability of IL-27 to activate IL27RA signaling was thus investigated in T-cell blasts of patients by flow cytometry and western blotting. STAT1 and STAT3 became rapidly phosphorylated from 2 minutes in lysates of control T cells in response to IL-27 and STAT1 and STAT3 phosphorylation persisted at 30 minutes (data not shown). Phospho-STAT1 and 3 proteins were also detectable by intracellular staining at 15 minutes of stimulation with IL-27 (data not shown). In contrast, phosphorylation of STAT1 and STAT3 was not or weakly detectable in IL-27-stimulated T cells blasts from P1.1 and P2 respectively (data not shown). Therefore, these data demonstrate that the genetic variants in IL27RA identified in the three patients are deleterious and behave as a loss-of-function.

Consequences of IL27RA Deficiency on T Cell Proliferation and Effector Differentiation We next assessed the functional consequences of IL27RA deficiency. Early studies showed that IL-27 could favor Th1 differentiation in mice⁹⁻¹². Accordingly, Th1-associated cytokines production of T cells from P1.1 and P1.2 was found to be impaired (data not shown), while numbers of Th1 were in normal ranges (data not shown). IL-27 is also known to promote T-cell proliferation of naïve CD4 and CD8+ T cells¹³⁻¹⁵. More recently expansion of memory-like CD8⁺ T cells was found IL-27 dependent in persistent viral infection in mice⁶. Therefore, we postulated that the robust expansion of EBV-specific T cells that is a key step in the immune control of EBV infection² could be IL-27 dependent. To assess this possibility, proliferation of T cells was analyzed upon stimulation with anti-CD3 antibody in the presence or not of IL-27 (data not shown). Addition of IL-27 alone did not induce any proliferation of control T cells, but it provided a synergistic effect on proliferation induced by low doses of anti-CD3 antibody (0.001-0.1 μg·mL⁻¹), an effect that is lost at the highest concentration of anti-CD3 antibody (10 μg·mL⁻¹) and is inhibited by addition of blocking anti-IL27 antibody (data not shown). Importantly, this synergistic effect of IL-27 was restricted to naïve T cells (data not shown). On the contrary, addition of IL-27 had no effect on CD3-induced proliferation of T cells from patients (P1.1 and P2) (data not shown). Furthermore, as expected from the critical role of STAT 1 in IL-27RA signaling and function, T cells from a patient with STAT 1-deficiency failed to proliferate in response to IL-27 (data not shown).

To formally prove that the defect in IL27RA directly impeded IL-27 function in T cells, T cells of P1.1 were transduced with a lentiviral vector (pLVX) containing a cDNA coding the wild-type IL27RA (pLVX-IL27RA) in order to restore the expression of IL27RA (data not shown). The pLVX plasmid also contains a mCherry reporter gene that allows to follow transduced cells. Transduction of P1.1 T cells with pLVX-IL27RA but not with an empty pLVX, restored IL27RA expression as assessed by western blot (data not shown). When IL27RA expression was restored in P1.1 T cells, cell lysates recovered detectable amounts of phosphorylated STAT1 and STAT3 in response to IL-27 stimulation. Phosphorylated STAT1 and STAT3 were also detected by intracellular staining in pLVX-IL27RA transduced mCherry⁺ patient T cells, but not in mCherry-(not transduced cells), whereas no phosphorylated STAT 1 and 3 were found in patient T cells transduced with an empty pLVX (data not shown). Synergistically enhanced CD3-dependent proliferation by IL-27 was also restored in mCherry⁺ patient T cells to a comparable level to that observed in control T cells, but not in mCherry⁻ patient T cells (data not shown). No such an effect was observed in cells transduced with the empty pLVX. Of note, overexpression of IL27RA in control T cells resulted in enhanced phosphorylation of STAT1 and STAT3 by IL-27, but it was not associated with an increase in proliferation. These data taken together indicate that IL-27 has the potency to sustain T-cell proliferation, while this synergistic effect is lost in patients with IL27RA deficiency.

This mechanism could therefore apply to the expansion and differentiation of (naïve) EBV-specific T cells. To address this question, we first screened PBMCs of P1.1 and P1.2 who both expressed class I MHC HLA-A02* molecules for the presence of EBV-specific T cells with HLA-A2 pentamers containing EBV peptides. EBV-specific T cells were detected in PBMCs of both P1.1 and P1.2 (data not shown), thus suggesting that IL-27 at first sight was not required for induction/expansion and survival of EBV-specific T cells. However, EBV-specific T cells of P1.1 and P1.2 failed to expand, in contrast to EBV-specific T cells from a HLA-A2-positive healthy individual, when co-cultured with irradiated autologous EBV-transformed B cells also named lymphoblastoid cell lines (LCLs) (data not shown). Furthermore, unlike control EBV-specific T cells, EBV-specific T cells of P1.1 and P1.2 expressed elevated levels of exhaustion and activation markers such as CD25, CD137, CD40L, PD1-L, KGRL1, CD57, LAG3 and 2B4 after 9 days of co-culture with LCLs (data not shown). Analysis of a blood sample from P1.1 at the time of the acute/severe infectious mononucleosis (SIM) showed EBV-specific T cells with a similar exhausted and activated phenotype confirming the data from in vitro expansion experiments. When tested, EBV-specific T cells from P1.1 exhibited a decreased capacity to kill autologous LCLs or HLA-A2-matched LCLs, compared to control EBV-specific T cells (data not shown). As P2 did not express class I MHC HLA-A02* molecules, we could not analyze EBV-specific T cells in P2 as only EBV-specific pentamers HLA-A2 are available. However, in vitro expansion of CD8⁺ T cells, which should contain most of the EBV-specific T cells were analyzed after co-culture of PBMCs from P2 with autologous LCLs. Expanded CD8⁺ T cells from P2 in contrast to control expanded CD8+ cells exhibited increased levels of activation and exhausted markers, like expanded EBV-specific T cells from P1.1 and P1.2 (data not shown).

Of note, LCLs of P1.1, P1.2 and P2 expressed similar levels of HLA-A2, CD137L and CD70 molecules to those found on control LCLs (data not shown), while EBV-specific of T cells from P1.1, P1.2 and P2 (at day 0) displayed CD137 and CD27 expression levels comparable to control cells (data not shown). These data exclude an interference between IL-27 and CD27-CD70 and CD137-CD137L pathways that are known to be important for an efficient expansion of EBV-specific T cells^{1,3}. Collectively, these data show that in the absence of IL27RA, proliferation and differentiation of EBV-specific T cells into potent effector T lymphocytes are impaired in association with an abnormal activation and exhausted-like phenotype of EBV-specific T cells. Thus, the absence of IL-27 signals may result in non-resolutive activation and accelerated exhaustion of T cells and less efficient control of EBV-infected B cells.

IL-27 is Produced by B Cells Upon EBV Infection IL-27 is a heterodimeric cytokine composed of the IL-27p28 (encoded by IL27) and the EBV-induced gene 3 (encoded by EBI3). IL-27 is mainly produced by antigen-presenting cells (APCs), namely macrophages, dendritic cells and B cells¹⁶⁻¹⁸. Interestingly, most of these observations were made in mice in the context of persistent viral infection, in particular B-cell derived IL-27 appeared to be important to control persistent LCMV infection¹⁷. Since EBI3 was initially identified in EBV-transformed B cell lines or LCLs¹⁹ and found to be expressed in EBV-positive lymphomas¹⁹⁻²¹, we thus hypothesized that B cells could be one major source of IL-27 upon infection by EBV. This was also supported by the IL27RA-dependent proliferation of EBV-specific T cells when co-cultured with LCLs (data not shown). We initially found that IL-27 expression was induced in 20-30% of B cells when stimulated with PMA plus ionomycin, together with IL-2 expression in a fraction of B cells (data not shown). Next, B cells were infected with a GFP-tagged EBV and analyzed for IL-27 production. In GFP-positive EBV-infected B cells, IL-27 expression was up-regulated, while in GFP-negative B cells, it was not (data not shown). Furthermore, IL-27 accumulated in the supernatant of PBMCs upon infection by EBV, as well as in the supernatants of LCLs that arose from long-term cultures of PBMCs infected by EBV (data not shown). Taken together, these results show that B cells when infected and immortalized by EBV are IL-27 producers. Therefore, this direct production of IL-27 by EBV-infected B cells may be involved in the initiation and the support of the expansion and differentiation of EBV-specific T cells into potent effector T cells.

### IL-27 is Required for B-Cell Growth of EBV-Transformed B Cells

In the course of these experiments, we noticed that EBV-transformed B cell lines (LCLs) derived from PBMCs of the three IL27RA-deficient patients grew slowly and did not expand as efficiently as control LCLs (FIG. 1a). These observations could suggest an additional unexpected role for IL-27 in promoting the growth of LCLs via an autocrine loop. Indeed, control LCLs were found to express IL27RA, while no expression of IL27RA was detectable on the surface of LCLs from P1.1, P1.2 and P.2 (data not shown). Notably, STAT 1 and STAT3 phosphorylations were detectable in control LCLs upon incubation with IL-27 indicating that IL27RA is functional in LCLs. In contrast, these phosphorylations were absent in IL-27-incubated IL27RA-deficient LCLs from P1.1, P1.2 and P2, as well as in LCLs derived from STAT1-deficient patients (data not shown). STAT1-deficient LCLs also failed to expand (FIG. 1a). The decreased expansion of IL27RA-deficient LCLs (and STAT1 LOF LCLs) was associated with a significant diminution of indexes of proliferation compared to those of control LCLs (data not shown). The low proliferation associated with the defective STAT1 phosphorylation of IL27RA-deficient LCLs were amended to levels comparable to those of controls LCLs when IL27RA expression was restored by transduction of the expression vector pLVX-IL27RA. No correction was observed in cells transduced with an empty pLVX vector (data not shown). Of note, IL27RA and STAT1-deficient LCLs produced IL-27 as control LCLs, excluding a defect in IL-27 production that could participate to their inability to grow (data not shown). These data demonstrate that the impaired proliferation and STAT1 phosphorylation of IL27RA-deficient LCLs directly result from IL27RA deficiency.

Furthermore, addition of blocking antibody to IL-27, but not to IL-2 significantly decreased cell growth of control LCLs (FIG. 1b and FIG. 2a) and abolished IL-27-induced STAT1 and STAT3 phosphorylation (FIG. 2b), which support a role of autocrine IL-27 production in LCLs expansion. Further underpinning this possibility, CRISPR-Cas9 genome editing in control LCLs that selectively inactivated IL27 by targeting exon 1 or exon 2 of IL27 resulted in rapid accumulation of DAPI⁺ dead cells in the cultures, whereas genome editing with an empty CRISPR-Cas9 vector did not affect cell viability (data not shown). As expected addition of exogenous IL-27 prevented cell-death of LCLs induced by inactivation of IL-27. However, cells failed to grow in these conditions. This may be explained by decreased IL27RA expression in the absence of IL-27; IL-27 when bound to IL27RA could be important to stabilize IL27RA at the cell surface expression and/or to activate IL27RA gene expression. This assumption was confirmed with the obtaining of control LCLs over-expressing ectopic IL27RA (after have being transduced with the pLVX-IL27RA), in which IL27 was inactivated by CRISPR-Cas9 resulting in decreased IL-27 production (data not shown). Contrary to control LCLs in which IL27 was inactivated, control LCLs overexpressing IL27RA (in which IL27 was inactivated) grew and expand when exogenous IL-27 was added (data not shown). Importantly, deprivation of exogenous IL-27 rapidly triggered cell death of these cells. No such an effect was observed in cells transduced with an empty CRISPR-Cas9 vector. Therefore, these results show that autocrine IL-27 production is required for cell growth of EBV-transformed B cells. Importantly, this could explain why in all three patients, severe infectious mononucleosis did not develop further into B-cell lymphoproliferation or lymphoma as it is observed in patients with immunocompromised T cell responses to EBV (such as patients with immunosuppressive treatments or suffering from an inherited immunodeficiency associated with a high susceptibility to EBV^{1,3}). Nevertheless, in vivo, during the initial infection, expansion of infected B cells appears not dependent on IL-27 since the three patients developed severe infectious mononucleosis. The initial EBV latency program that is activated during primary infection could be sufficient to sustain B-cell proliferation independently of IL-27. Further investigations are required to decipher the precise role of IL-27 in proliferation and maintenance of EBV-infected B cells.

Identification of Neutralizing Anti-IL-27 Autoantibodies in Patients with EBV Viral Diseases

Neutralizing anti-cytokine autoantibodies have been identified in several infectious and immunological conditions causing immunodeficiency and/or immune dysregulation²². Some of these anti-cytokine autoantibodies have been shown to phenocopy genetically determined immunodeficiencies such as defects in IL-17A/F^{23,24} and IL-6^{25,26}. Hence, we hypothesized that anti-IL-27 autoantibodies could phenocopy IL27RA deficiency and may explain sporadic acute EBV-driven infectious mononucleosis as observed in adolescents and adults. We set up an ELISA to detect anti-IL-27 autoantibodies to firstly screen sera of patients with autoimmune and/or inflammatory diseases including inborn errors of immunity previously associated with high titers of various anti-cytokine autoantibodies such as AIRE deficiency known to cause autoimmune polyendocrinopathy candidiasis ectodermal dystrophy (APECED) syndrome and STAT1 gain-of-function (GOF) mutations (data not shown). High titers of autoantibodies that positively reacted with recombinant IL-27 in ELISA were found in most patients with STAT1 GOF and correlated with the presence of detectable serum concentrations of IL-27 in these patients (data not shown). In contrast, the titers in patients with APECED titers were not significantly different from those detected in healthy controls that were very low or undetectable. We next tested sera of patients with diverse EBV-related conditions including EBV+ B-cell lymphoma, acute infectious mononucleosis (IM) and chronic EBV active infection, a severe viral disease characterized by persistent infection of T and/or NK cells^{27,28}. Strikingly, significant high titers of anti-IL-27 antibodies were detected in patients with IM and CAEBV in association with detectable levels of plasma IL-27 like in STAT1 GOF patients. In patients with EBV+ lymphoma that mostly include Hodgkin lymphoma, titers of anti-IL-27 antibodies were lower, while levels of IL-27 were comparable to those detected in patients with IM and CAEBV. Consistent with the ELISA data, sera from patients with infectious mononucleosis, STAT1 GOF mutations were able to detect recombinant IL-27 (data not shown), while serum from healthy control donors (data not shown) did not detect recombinant IL-27. These results are thus consistent with the presence of autoantibodies specific to IL-27 in patients with infectious mononucleosis and STAT1 GOF mutations. Importantly, these anti-IL27 autoantibodies (from both patients with IM and STAT1 GOF) exerted a specific neutralizing activity since they were able to inhibit phospho-STAT1 induction (data not shown) and synergistic CD3-dependent proliferation triggered by IL-27 in T cells (data not shown), whereas a control serum caused no inhibition. Collectively, these data demonstrate the presence of anti-IL-27 autoantibodies with neutralizing capacity in patients with IM, CAEBV and STAT1 GOF.

### Discussion

We report a novel primary immunodeficiency characterized by high susceptibility to herpes virus, mostly EBV revealing the importance of the IL-27 response in anti-EBV immunity. A role of IL-27 in anti-viral immunity in humans has been previously suspected. It is herein shown that IL-27 is required for the expansion of naive EBV-specific T cells and their differentiation into potent effector T cells. IL-27 can be viewed as providing an additional costimulatory signal that contributes to the required major amplification of the T-cell immune response against infected B cells as it is the case for other co-stimulatory pathways, i.e., SLAM, CD27 and CD137¹. Indeed, vigorous expansion of EBV-specific T cells is needed to efficiently control and eliminate EBV-infected cells. Notably, EBV-specific T cells may represent up to 40% of circulating T cells in healthy individuals during EBV primo infection^{29,30}. The critical protective role of IL-27 in the immune control to EBV is also supported by the presence of anti-IL-27 autoantibodies that interfere with IL-27 in individuals presenting acute sporadic severe infectious mononucleosis, thus possibly phenocopying patients with IL27RA deficiency. Interestingly, patients with CAEBV also exhibit high titers of anti-IL-27 autoantibodies. In contrast to infectious mononucleosis, CAEBV diseases are characterized by EBV-infected T and/or cells associated with high blood loads of EBV that persist over years. Pathophysiological mechanisms of CAEBV remain largely unknown. Anti-IL-27 autoantibodies may play a role in the persistence of EBV infection in individuals with CAEBV. As it was shown for other anti-cytokine autoantibodies, it can be postulated that anti-IL-27 autoantibodies preexist infection with EBV in individual patients underlying infectious mononucleosis once infected by EBV²². Patients with immune deficiency caused by STAT1 GOF also exhibited high levels of anti-IL-27 autoantibodies although these patients are not particularly susceptible to EBV infection. However, in these patients, effector T cell functions are exacerbated possibly compensating the defective IL-27 response. Interestingly, detectable levels of IL-27 (that can be high) were detected in the serum of patients with sporadic IM or STAT1 GOF and correlated with high titers of anti-IL-27 autoantibodies. These high levels of IL-27 might preexist to autoantibodies and trigger their occurrence or/and might represent a compensatory mechanism to bypass the blocking effect of anti-IL-27 autoantibodies. Finally, the observation that IL-27 is involved in the growth of EBV-transformed B cells could explain the self-limiting phenotype of EBV infection in IL27RA deficient patients and suggests that EBV in vivo “captures” the IL-27 pathway to its own advantage by providing a selective advantage to infected B cells. IL-27 could thus be viewed as a novel therapeutic target to inhibit growth of EBV+ B cells in immunocompromised individuals or in patients with EBV+ lymphomas. Importantly, this hypothesis is further supported by the fact that patients with EBV+ lymphomas had less or no detectable autoantibodies to IL-27 (in contrast to patients with sporadic EBV+ IM), while exhibiting detectable serum levels of IL-27 (comparable to those of patients with sporadic EBV+ IM). In conclusion, this reports pinpoints to a double role of IL-27 as a costimulatory molecule for T cell proliferation, differentiation into effector cells required for the control of EBV infection and as a proliferation and/or survival signal of EBV-infected B cells, an observation that deserves further study to assess its mechanism.

### REFERENCES

Throughout this application, various references describe the state of the art to which this invention pertains. The disclosures of these references are hereby incorporated by reference into the present disclosure.
1 Latour, S. & Fischer, A. Signaling pathways involved in the T-cell-mediated immunity against Epstein-Barr virus: Lessons from genetic diseases. Immunol Rev 291, 174-189, doi:10.1111/imr.12791 (2019).
2 Taylor, G. S., Long, H. M., Brooks, J. M., Rickinson, A. B. & Hislop, A. D. The immunology of Epstein-Barr virus-induced disease. Annu Rev Immunol 33, 787-821, doi:10.1 146/annurev-immunol-032414-112326 (2015).
3 Tangye, S. G. & Latour, S. Primary immunodeficiencies reveal the molecular requirements for effective host defense against EBV infection. Blood 135, 644-655, doi:10.1182/blood.2019000928 (2020).
4 Hirahara, K. et al. Asymmetric Action of STAT Transcription Factors Drives Transcriptional Outputs and Cytokine Specificity. Immunity 42, 877-889, doi:10.1016/j.immuni.2015.04.014 (2015).
5 Kastelein, R. A., Hunter, C. A. & Cua, D. J. Discovery and biology of IL-23 and IL-27: related but functionally distinct regulators of inflammation. Annu Rev Immunol 25, 221-242, doi:10.1146/annurev.immunol.22.012703.104758 (2007).
6 Huang, Z. et al. IL-27 promotes the expansion of self-renewing CD8(+) T cells in persistent viral infection. J Exp Med 216, 1791-1808, doi:10.1084/jem.20190173 (2019).
7 Sprecher, C. A. et al. Cloning and characterization of a novel class I cytokine receptor. Biochem Biophys Res Commun 246, 82-90, doi:10.1006/bbrc.1998.8576 (1998).
8 Pflanz, S. et al. WSX-1 and glycoprotein 130 constitute a signal-transducing receptor for IL-27. J Immunol 172, 2225-2231, doi:10.4049/jimmunol.172.4.2225 (2004).
9 Chen, Q. et al. Development of Th1-type immune responses requires the type I cytokine receptor TCCR. Nature 407, 916-920, doi:10.1038/35038103 (2000).
10 Owaki, T. et al. A role for IL-27 in early regulation of Th1 differentiation. J Immunol 175, 2191-2200, doi:10.4049/jimmunol.175.4.2191 (2005).
11 Yoshida, H. & Hunter, C. A. The immunobiology of interleukin-27. Annu Rev Immunol 33, 417-443, doi:10.1146/annurev-immunol-032414-112134 (2015).
12 Artis, D. et al. The IL-27 receptor (WSX-1) is an inhibitor of innate and adaptive elements of type 2 immunity. J Immunol 173, 5626-5634, doi:10.4049/jimmunol.173.9.5626 (2004).
13 Schneider, R., Yaneva, T., Beauseigle, D., El-Khoury, L. & Arbour, N. IL-27 increases the proliferation and effector functions of human naive CD8+ T lymphocytes and promotes their development into Tcl cells. Eur J Immunol 41, 47-59, doi:10.1002/eji.201040804 (2011).
14 Charlot-Rabiega, P., Bardel, E., Dietrich, C., Kastelein, R. & Devergne, O. Signaling events involved in interleukin 27 (IL-27)-induced proliferation of human naive CD4+ T cells and B cells. J Biol Chem 286, 27350-27362, doi:10.1074/jbc.M111.221010 (2011).
15 Pflanz, S. et al. IL-27, a heterodimeric cytokine composed of EBI3 and p28 protein, induces proliferation of naive CD4+ T cells. Immunity 16, 779-790, doi:10.1016/s1074-7613(02) 00324-2 (2002).
16 Harker, J. A. et al. Interleukin-27R Signaling Mediates Early Viral Containment and Impacts Innate and Adaptive Immunity after Chronic Lymphocytic Choriomeningitis Virus Infection. J Virol 92, doi:10.1128/JVI.02196-17 (2018).
17 Pratumchai, I. et al. B cell-derived IL-27 promotes control of persistent LCMV infection. Proc Natl Acad Sci USA 119, doi:10.1073/pnas.2116741119 (2022).
18 Devergne, O., Birkenbach, M. & Kieff, E. Epstein-Barr virus-induced gene 3 and the p³⁵ subunit of interleukin 12 form a novel heterodimeric hematopoietin. Proc Natl Acad Sci USA 94, 12041-12046, doi:10.1073/pnas.94.22.12041 (1997).
19 Devergne, O. et al. A novel interleukin-12 p40-related protein induced by latentEpstein-Barr virus infection in B lymphocytes. J Virol 70, 1143-1153, doi:10.1128/JVI.70.2.1143-1153.1996 (1996).
20 Niedobitek, G., Pazolt, D., Teichmann, M. & Devergne, O. Frequent expression of the Epstein-Barr virus (EBV)-induced gene, EBI3, an IL-12 p40-related cytokine, in Hodgkin and Reed-Sternberg cells. J Pathol 198, 310-316, doi:10.1002/path.1217 (2002).
21 Larousserie, F. et al. Analysis of interleukin-27 (EBI3/p28) expression in Epstein-Barr virus- and human T-cell leukemia virus type 1-associated lymphomas: heterogeneous expression of EBI3 subunit by tumoral cells. Am J Pathol 166, 1217-1228, doi:10.1016/S0002-9440(10) 62340-1 (2005).
22 Puel, A., Bastard, P., Bustamante, J. & Casanova, J. L. Human autoantibodies underlying infectious diseases. J Exp Med 219, doi:10.1084/jem.20211387 (2022).
23 Kisand, K. et al. Chronic mucocutaneous candidiasis in APECED or thymoma patients correlates with autoimmunity to Th17-associated cytokines. J Exp Med 207, 299-308, doi:10.1084/jem.20091669 (2010).
24 Puel, A. et al. Autoantibodies against IL-17A, IL-17F, and IL-22 in patients with chronic mucocutaneous candidiasis and autoimmune polyendocrine syndrome type I. J Exp Med 207, 291-297, doi:10.1084/jem.20091983 (2010).
25 Nanki, T. et al. Suppression of elevations in serum C reactive protein levels by anti-IL-6 autoantibodies in two patients with severe bacterial infections. Ann Rheum Dis 72, 1100-1102, doi:10.1136/annrheumdis-2012-202768 (2013).
26 Puel, A. et al. Recurrent staphylococcal cellulitis and subcutaneous abscesses in a child with autoantibodies against IL-6. J Immunol 180, 647-654, doi:10.4049/jimmunol.180.1.647 (2008).
27 Fournier, B. & Latour, S. Immunity to EBV as revealed by immunedeficiencies. Curr Opin Immunol 72, 107-115, doi:10.1016/j.coi.2021.04.003 (2021).
28 Kawamoto, K. et al. A distinct subtype of Epstein-Barr virus-positive T/NK-cell lymphoproliferative disorder: adult patients with chronic active Epstein-Barr virus infection-like features. Haematologica 103, 1018-1028, doi:10.3324/haematol.2017.174177 (2018).
29 Hoshino, Y., Nishikawa, K., I to, Y., Kuzushima, K. & Kimura, H. Kinetics of Epstein-Barr virus load and virus-specific CD8+ T cells in acute infectious mononucleosis. J Clin Virol 50, 244-246, doi:10.1016/j.jcv.2010.11.017 (2011).

30 Callan, M. F. et al. Direct visualization of antigen-specific CD8+ T cells during the primary immune response to Epstein-Barr virus In vivo. J Exp Med 187, 1395-1402, doi:10.1084/jem.187.9.1395 (1998).
31 Izawa, K. et al. Inherited CD70 deficiency in humans reveals a critical role for the CD70-CD27 pathway in immunity to Epstein-Barr virus infection. J Exp Med 214, 73-89, doi:10.1084/jem.20160784 (2017).
32 Martin, E. et al. CTP synthase 1 deficiency in humans reveals its central role in lymphocyte proliferation. Nature 510, 288-292, doi:10.1038/nature13386 (2014).
33 McStay, G. P., Salvesen, G. S. & Green, D. R. Overlapping cleavage motif selectivity of caspases: implications for analysis of apoptotic pathways. Cell Death Differ 15, 322-331, doi:10.1038/sj.cdd.4402260 (2008).

1. A method of treating an EBV-driven B lymphoproliferative disease in patient in need thereof comprising administering to the patient a therapeutically effective amount of an IL-27 antagonist.

2. The method of claim 1 wherein the B lymphoproliferative disease is selected from the group consisting of non-Hodgkin's lymphoma, Burkitt's lymphoma, small lymphocytic lymphoma, primary effusion lymphoma, diffuse large B-cell lymphoma, splenic marginal zone lymphoma, MALT (mucosa-associated lymphoid tissue) lymphoma, hairy cell leukemia, chronic lymphocytic leukemia, B-cell prolymphocytic leukemia, B cell lymphomas, leukemias, myelomas, small lymphocytic lymphoma, B cell prolymphocytic leukemia, lymphoplasmacytic lymphoma, splenic marginal zone lymphoma, plasma cell myeloma, solitary plasmacytoma of bone, extraosseous plasmacytoma, extra-nodal marginal zone B cell lymphoma of mucosa-associated (MALT) lymphoid tissue, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma/leukemia, grey zone lymphoma, B cell proliferations of uncertain malignant potential, lymphomatoid granulomatosis, and post-transplant lymphoproliferative disorder.

3. The method of claim 1 wherein the EBV-driven B lymphoproliferative disease is a B-cell lymphoma.

4. The method according to claim 1, wherein the IL-27 antagonist is selected from the group consisting of antibodies that bind IL-27 heterodimer, antibodies that bind p28, antibodies that bind EBI3, antibodies that bind IL-27R heterodimer, antibodies that bind IL-27RA, IL-27RA extracellular domains (ECDs), and IL-27RA ECD fusion molecules.

5. The method of claim 4 wherein the IL-27 antagonist is an antibody that binds to p28, but does not bind to EBI3.

6. The method of claim 4 wherein the IL-27 antagonist is an antibody that binds to EBI3, but does not bind to p28.

7. The method of claim 4 wherein the IL-27 antagonist is an IL-27RA antibody.

8. The method of claim 2, wherein the B cell lymphoma is Hodgkin's disease, B cell non-Hodgkin's lymphoma (NHL), Waldenstrom's macroglobulinaemia or a central nervous system lymphomas.

9. The method of claim 2, wherein the leukemia is acute lymphoblastic leukemia (ALL), chronic lymphocytic leukemia (CLL), hairy cell leukemia, or chronic myoblastic leukemia.

10. The method of claim 2, wherein the myeloma is multiple myeloma.
